(19) United States
(12) Patent Application Publication
(10) Pub. No.: US 2025/0019323 A1
(43) Pub. Date: Jan. 16, 2025

(54) CATALYST AND METHOD FOR LOW-TEMPERATURE OXIDATIVE DEHYDROGENATION OF LOW-CARBON ALKANES TO LIGHT OLEFINS

(71) Applicant: Braskem America, Inc., Philadelphia, PA (US)

(72) Inventors: Ishant Khurana, Philadelphia, PA (US); Graham Gregorich, Philadelphia, PA (US); Scott Mitchell, Philadelphia, PA (US)

(73) Assignee: Braskem America, Inc., Philadelphia, PA (US)

(21) Appl. No.: 18/749,961

(22) Filed: Jun. 21, 2024

Related U.S. Application Data
(60) Provisional application No. 63/522,195, filed on Jun. 21, 2023.

(51) Int. Cl. C07C 5/32; B01J 21/12; B01J 29/072; B01J 37/00; B01J 37/04; B01J 37/08

(52) U.S. Cl. CPC C07C 5/324; B01J 21/12; B01J 29/072; B01J 37/0036; B01J 37/04; B01J 37/08; C07C 2523/72; C07C 2529/06

(57) ABSTRACT

A heterogeneous catalyst composition may include a metal catalyst supported on a porous mixed metal oxide support. A process for catalytic oxidative dehydrogenation of hydrocarbons may include contacting, in a reactor system, a hydrocarbon-containing feedstock with the heterogeneous catalyst composition to generate olefinic compounds. A process for preparing a heterogeneous catalyst composition may include combining a porous mixed metal oxide support with at least one metal catalyst precursor to form a catalyst precursor mixture, wherein the at least one metal catalyst precursor comprises at least one metal compound selected from the group consisting of transition metal compounds, rare-earth metal compounds, or a mixture thereof, and heating the catalyst precursor mixture to a temperature of about 390° C. to about 750° C. to form a heterogeneous catalyst composition.

### BACKGROUND

Light olefins, ethylene, and propylene are important raw materials for producing polyolefins such as polyethylene (PE) and polypropylene (PP). A common technology to produce light olefins is the non-oxidative dehydrogenation of light alkanes. Still, it is thermodynamically limited and highly energy- and capital-intensive, which prompted the exploration of the new process intensifying opportunities in the form of catalytic oxidative dehydrogenation (ODH). While this emerging oxidative dehydrogenation (ODH) process is thermodynamically favored and more energy efficient (exothermic), it suffers from poor product selectivity limiting the olefin yields. Additionally, catalysts with acceptable activity tend to over-oxidize the olefin and have unacceptably large selectivities to CO and CO₂. Thus, the challenge in this path to process intensification lies in poor product selectivity in the presence of oxygen at moderate temperatures (>400° C.).

Hence, there exists a need for a catalyst with superior catalyst reactivity, product selectivity, and yield, while also enabling the ODH of light alkanes to operate at a relatively lower temperature (<400° C.), further making it more energy and cost-efficient.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a heterogeneous catalyst composition that includes a metal catalyst supported on a porous mixed metal oxide support.

In another aspect, embodiments disclosed herein relate to a process for catalytic oxidative dehydrogenation of hydrocarbons that includes contacting, in a reactor system, a hydrocarbon-containing feedstock with a heterogeneous catalyst composition to generate olefinic compounds. The heterogeneous catalyst composition includes a metal catalyst supported on a porous mixed metal oxide support.

In yet another aspect, embodiments disclosed herein relate to a process for preparing a heterogeneous catalyst composition, including combining a porous mixed metal oxide support with at least one metal catalyst precursor to form a catalyst precursor mixture, wherein the at least one metal catalyst precursor comprises at least one metal compound selected from the group consisting of transition metal compounds, rare-earth metal compounds, or a mixture thereof, and heating the catalyst precursor mixture to a temperature of about 390° C. to about 750° C. to form a heterogeneous catalyst composition.

Other aspects and advantages of the claimed subject matter will be apparent from the following description and the appended claims.

### BRIEF DESCRIPTION OF DRAWINGS

FIG. 1 shows catalyst selectivity and reactivity versus temperature data for Example 1.

### DETAILED DESCRIPTION

Embodiments disclosed herein relate to a heterogeneous catalyst composition, a process to prepare the heterogeneous catalyst composition and a process for oxidative dehydrogenation (ODH) of hydrocarbons to form olefinic monomers using the heterogeneous catalyst composition. In particular, embodiments described herein are directed to a heterogeneous catalyst containing a metal catalyst, particularly a transition and/or rare earth metal incorporated onto a porous mixed metal oxide support. The heterogeneous catalyst composition may be used in a process for ODH of hydrocarbons to form olefinic monomers with improved reactivity, product selectivity, and yield by using a chemical looping reactor system, while also enabling the ODH process to operate at a relatively lower temperature compared to commercially available ODH technology. Embodiments described herein also relate to a process to prepare the heterogeneous composition either inside or outside a reactor system.

### Heterogeneous Catalyst Composition

One or more embodiments relate to a heterogeneous catalyst composition containing a metal catalyst supported on a porous mixed metal oxide support.

The heterogeneous catalyst composition may contain a metal catalyst, such as a transition metal compound, a rare-earth metal compound, or a mixture thereof. Examples of metal compounds may include at least one transition metal compound, where the transition metal belongs to groups 4-12 in the periodic table of elements, including but not limited to elements V, Cr, Mn, Fe, Co, Ni, Cu, Mo, Zn, W, and mixtures thereof, with the metal being in the oxidized state or any transition state.

In one or more embodiments, the metal catalyst is in the oxidized metal form (i.e., one or more of its cationic forms) and it is anchored into the mixed metal oxide support. For the purposes of the present disclosure, the metal catalyst may be in the oxidized state or any transition state.

In one or more embodiments, the metal catalyst comprises a metal compound selected from the group consisting of Cu, Fe, Zn, or any combination thereof. In particular embodiments, such metals may be incorporated into a porous mixed metal oxide support that comprises silicon oxide and aluminum oxide.

The heterogeneous catalyst composition may contain a porous mixed metal oxide support, such as a mixture of oxides selected from silicon oxide, aluminum oxide, boron oxide, gallium oxide, and mixtures thereof.

One or more embodiments may include a mixture of silicon oxide and another metal oxide, such as aluminum oxide, boron oxide, gallium oxide, and mixtures thereof as the porous mixed metal oxide support for the heterogeneous catalyst composition. The porous mixed metal oxide support for the heterogeneous catalyst composition may contain a mole ratio of silicon oxide to an oxide selected from aluminum oxide, boron oxide, gallium oxide, and mixtures thereof of greater than about 10:1. For example, the mole ratio may be in a range having a lower limit of about 10:1, 10.2:1, and 10.3:1 and an upper limit of about 10.5:1, 13:1, and 15:1, where any lower limit and upper limit may be used in combination.

One or more embodiments may include a zeolite as the porous mixed metal oxide support for the heterogeneous catalyst composition. The zeolite may be mordenite (MOR), Zeolite Socony Mobil-5 (ZSM-5), beta zeolites (BEA), or other zeolite frameworks.

The heterogeneous catalyst composition may contain a catalyst loading, or the loading of the metal-supported onto the porous mixed metal oxide, ranging from about 1% to about 5% by weight, based on the total weight of the heterogeneous catalyst composition. For example, the loading of metal supported onto the porous mixed metal oxide may be in a range having a lower limit of about 1, 1.5, 2.0, 2.5, or 3% by weight, and an upper limit of about 3.5, 4, 4.5 or 5% by weight, based on the total weight of the heterogeneous catalyst composition, where any lower limit and upper limit may be used in combination.

The heterogeneous metal catalyst may have a porosity ranging from 0.10 to 0.20 cm³/g of catalyst, preferably from 0.12 to 0.20 cm³/g of catalyst. The porosity of the catalyst may be measured by BET analysis.

### Preparation of Heterogeneous Catalyst Composition

One or more embodiments disclosed herein relate to a process to prepare a heterogeneous catalyst composition. Specifically, one or more embodiments relate to the preparation of a heterogeneous catalyst composition from a porous mixed metal oxide and at least one metal catalyst precursor to form a catalyst precursor mixture. Preparation of the heterogeneous catalyst composition may also include heating the catalyst precursor mixture. The heterogeneous catalyst composition may be prepared either inside or outside a reactor system.

It is envisioned that the metal catalyst precursor mixture may include the metal to be incorporated into the catalyst (i.e., the metal catalyst) in the form of an oxide, carbonate, nitrate, or acetate, with the metal being in any transition state. Examples of forms taken by the metal catalyst precursor may include vanadium oxides, chromium oxides, manganese oxides, iron oxides, cobalt oxides, nickel oxides, copper oxides, molybdenum oxides, zinc oxides, tungsten oxides, vanadium carbonates, chromium carbonates, manganese carbonates, iron carbonates, cobalt carbonates, nickel carbonates, copper carbonates, molybdenum carbonates, zinc carbonates, tungsten carbonates, vanadium nitrates, chromium nitrates, manganese nitrates, iron nitrates, cobalt nitrates, nickel nitrates, copper nitrates, molybdenum nitrates, zinc nitrates, tungsten nitrates, vanadium acetates, chromium acetates, manganese acetates, iron acetates, cobalt acetates, nickel acetates, copper acetates, molybdenum acetates, zinc acetates, tungsten acetates or mixtures thereof.

In one or more embodiments, the heterogeneous catalyst composition is prepared in a reactor system. The reactor system may include a tubular reactor, a continuous stirred tank reactor (CSTR), or a loop reactor.

In one or more embodiments, the reactor system is operated as a continuous process, a semi-continuous process, or a batch process.

In one or more embodiments, the reactor system includes a single reactor. In some embodiments, the reactor system includes at least a first reactor and a second reactor connected in a continuous loop for catalyst circulation.

In one or more embodiments, a heterogeneous catalyst composition is prepared from a porous mixed metal oxide and at least one metal catalyst precursor to form a catalyst precursor mixture.

The porous mixed metal oxide included in the process may include the porous mixed metal oxides as previously described.

In one or more embodiments, the catalyst precursor mixture includes at least one metal compound selected from the group consisting of transition metal compounds, rare-earth metal compounds, or a mixture thereof. The at least one metal compound may include any of the metal compounds as previously described.

The process to prepare the heterogeneous catalyst composition may be carried out either outside or inside the reactor system. In one or more embodiments, preparing the heterogeneous catalyst composition outside or inside the reactor system includes combining a porous mixed metal oxide with a metal catalyst precursor to form a catalyst precursor mixture and heating the catalyst precursor mixture to a temperature of about 390° C. to about 750° C. to form the at least one heterogeneous catalyst composition. In one or more embodiments, the heating may occur at a temperature of about 350° C. to about 450° C.

### Process for Oxidative Dehydrogenation of Hydrocarbons

One or more embodiments disclosed herein relate to a process for ODH of hydrocarbons to form olefinic compounds (also referred to as monomers) using at least one heterogeneous catalyst composition. The process may include contacting, in a reactor system, a hydrocarbon-containing feedstock and an oxygen source with the at least one heterogeneous catalyst composition to generate olefinic compounds.

The reactor system used in the process for ODH of hydrocarbons to form olefinic compounds or olefinic monomers may include any of the reactor systems as previously described.

As described above, the reactor system may include a tubular reactor, a continuous stirred tank reactor (CSTR), or a loop reactor and the reactor system may be operated as a continuous process, a semi-continuous process, or a batch process. The reactor system may include at least a first reactor and a second reactor, where the first and second reactors are connected in a continuous loop for catalyst circulation.

In some embodiments, the reactor system includes a single reactor. In embodiments where the process for ODH of hydrocarbons to form olefinic compounds or olefinic monomers is carried out in a single reactor, the at least one heterogeneous catalyst composition is contacted sequentially: first with a hydrocarbon-containing feedstock, then with an oxygen source.

The process for ODH of hydrocarbons to form olefinic compounds may include contacting at least one heterogeneous catalyst composition with a hydrocarbon-containing feedstock. The hydrocarbon-containing feedstock may include a refinery range hydrocarbon. In one or more embodiments, the refinery range hydrocarbon includes at least one light alkane, for example, ethane, propane, n-butane, isobutene, n-pentane, isopentane, n-hexane, isohexane, and combinations therein.

In some embodiments, the hydrocarbon-containing feedstock optionally contains a diluent. The diluent may include nitrogen (N₂), argon (Ar), or helium (He).

In one or more embodiments, the at least one heterogeneous catalyst composition is prepared outside of the reactor system. In this case, the process for catalytic oxidative dehydrogenation of hydrocarbons may include preparing the at least one heterogeneous catalyst composition outside of the reactor system and loading the at least one heterogeneous catalyst composition into the reactor system.

In one or more embodiments, the at least one heterogeneous catalyst composition is prepared inside of the reactor system. In this case, the process for catalytic oxidative dehydrogenation of hydrocarbons may include preparing the at least one heterogeneous catalyst composition may include loading a catalyst precursor mixture into the reactor system and heating at a temperature of about 390° C. to about 750° C.

The process for ODH of hydrocarbons to form olefinic compounds may include contacting at least one heterogeneous catalyst composition with an oxygen source. In one or more embodiments, the oxygen source includes a purified oxygen (O₂) stream, an air stream, or a mixture thereof. In some embodiments, the oxygen source optionally contains a diluent. The diluent may include carbon dioxide (CO₂), nitrogen (N₂), argon (Ar), or helium (He).

In one or more embodiments, the process for ODH of a hydrocarbon feedstock to form olefinic compounds using at least one heterogeneous catalyst composition is an exothermic process. In one or more embodiments, the contacting in a reactor system is conducted at a temperature of about 750° C. or less, or of about 450° C., or of about 400° C. or less and/or a pressure of about 20 atm or less. For example, the contacting process temperature may be in a range having a lower limit of about 0, 50, 100, 150, 200, 250, 300, 350, 400, 450, 500, and 750° C. and an upper limit of about 400, 450, 500, 550, 600, 650, 700, 740, 745, and 750° C., where any lower limit and the upper limit may be used in combination. Additionally, for example, the contacting process pressure may be in a range having a lower limit of about 0, 1, 5, 10, 15, and 20 atm and an upper limit of about 19, 19.5, and 20 atm, where any lower limit and the upper limit may be used in combination.

In one or more embodiments, the selectivity of the process of the present disclosure towards olefinic compounds may range from 50% to 100%. For example, the selectivity of the process may be in a range having a lower limit of about 50, 55, 60, 65, 70, 75 or 80% and an upper limit of about 85%, 90%, 95% or 100% where any lower limit and the upper limit may be used in combination.

In one or more embodiments, the conversion of hydrocarbon-containing feedstock in the process according to the present disclosure ranges from 1% to 10%.

In one or more embodiments, the reactivity of the catalyst/process according to the present disclosure ranges from 0.05 to 1.00, in mol product mol metal⁻¹h⁻¹

In one or more embodiments, the Gas Hourly Space Velocity (GHSV) to be applied to the process of the present disclosure may range from 3500 h⁻¹ to 20,000 h⁻¹, relative to the total gas feed.

The olefinic compound produced by the process for ODH of hydrocarbons described herein may include light olefins, α-olefins, and terminal dienes. Some examples of olefinic compounds may include ethene, propene, 1-butene, 2-methyl-but-1-ene, 1-n-pentene, 1-n-hexene, 2-methyl-pent-1-ene, 3-methyl-pent-1-ene, 1,3-butadiene, 1,3-pentadiene, 1,4-pentadiene, 1,3-hexadiene, 1,4-hexadiene, or 1,5-hexadiene.

### Example

The following example is provided for the purpose of further illustrating embodiments described herein and is in no way to be taken as limiting.

Example 1 is a catalyst prepared by the following steps. A 2.5 wt. % Cu-zeolite catalyst was prepared by physically mixing the required amounts of Cu(NO₃)₂(H₂O)ₓ and zeolite powder (with 5-10 mL of water for better homogenization) and grinding manually in a mortar and pestle for about 10-15 min. The obtained blue-colored solid paste was dried in an oven at 90° C. overnight. This was followed by heating in air at 600° C. for 6 h at 1° C. min⁻¹.

Selective Production of Propylene from Propane

A catalyst bed contained in a lab-scale, plug flow reactor was loaded with 0.2 g of example 1. A propane gas stream containing 20 vol % propane in 80 vol % N₂ gas was fed into the reactor at a rate of about 200 mL/min. The catalyst bed was maintained at a temperature from about 300° C. to about 400° C. The temperature of the catalyst bed was varied within the temperature range of about 300-400° C. and propylene selectivity was measured, along with the conversion rate of propane to propylene. The propylene selectivity is defined as (moles of propylene formed)/(total moles of products), and the reactivity towards propylene is defined as: (moles of propylene, out)/((moles of metal in the catalyst)*(residence time)). The product composition was analyzed using an Agilent 8890 gas chromatograph equipped with a thermal conductivity detector (TCD) and flame ionization detector (FID). The identity of the compounds was determined based on the order of elution following the boiling point of the solutes/compounds (FID) and thermal conductivity differences (TCD). The peaks areas obtained by integrating peaks on the gas chromatogram were used in the quantification of hydrocarbon compounds like propane, propylene, etc. (FID signal) and permanent gases like CO, CO2, etc. (TCD signal). More specifically, the peak areas were converted to molar concentrations using the molar response factors (TCD and FID) of various alkane and alkene products estimated from GC calibration with the refinery gas standard sample.

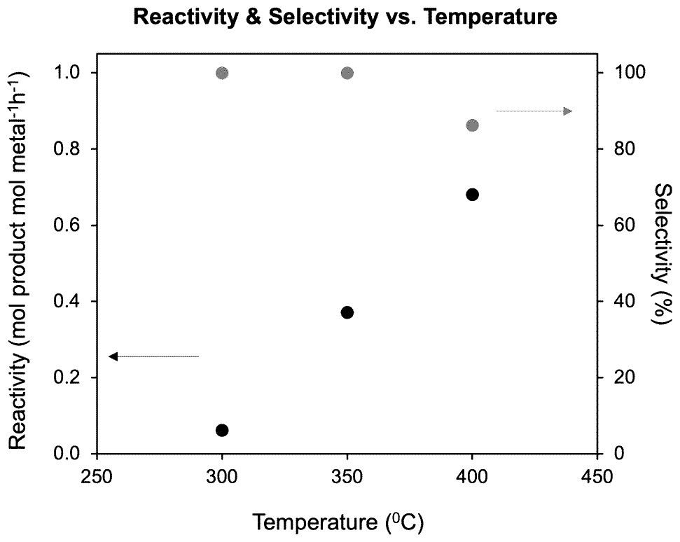

Results are shown in FIG. 1, which illustrates that the example 1 catalyst selectively converts propane at low temperatures and produces propylene as the main product Other side products include ethylene, CO, CO₂, H₂O. FIG. 1 also shows that the reactivity towards propylene (mol propylene mol metal⁻¹h⁻¹) increases with increasing temperature. Additionally, the selectivity is 100% when the catalyst bed is maintained at a temperature of 300° C. and 350° C., and the selectivity decreases to around 80% when the catalyst bed is maintained at a temperature of 400° C.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail, and a screw may be equivalent structures. It is the express intention of the applicant not to invoke 35 U.S.C. § 112 (f) for any limitations of any of the claims herein, except for those in which the claim expressly uses the words ‘means for’ together with an associated function.

What is claimed:

1. A heterogeneous catalyst composition comprising a metal catalyst supported on a porous mixed metal oxide support.

2. The heterogeneous catalyst composition of claim 1, wherein the metal catalyst comprises at least one metal compound selected from transition metal compounds, rare-earth metal compounds, or a mixture thereof.

3. The heterogeneous catalyst composition of claim 1, wherein the porous mixed metal oxide support comprises a mixture of oxides selected from silicon oxide, aluminum oxide, boron oxide, gallium oxide, and mixtures thereof.

4. The heterogeneous catalyst composition of claim 1, wherein the porous mixed metal oxide support is a zeolite.

5. The heterogeneous catalyst composition of claim 3, wherein the porous mixed metal oxide support comprises a mole ratio of silicon oxide to an oxide selected from aluminum oxide, boron oxide, gallium oxide, and mixtures thereof of greater than about 10:1.

6. The heterogeneous catalyst composition of claim 2, wherein the metal catalyst comprises at least one transition metal compound belonging to groups 4-12 in the periodic table of elements selected from the group consisting of V, Cr, Mn, Fe, Co, Ni, Cu, Mo, Zn, and W.

7. The heterogeneous catalyst composition of claim 1, wherein the metal catalyst comprises a metal compound selected from the group consisting of Cu, Fe, Zn, or combinations thereof, and the porous mixed metal oxide support comprises silicon oxide and aluminum oxide.

8. A process for catalytic oxidative dehydrogenation of hydrocarbons, the process comprising:
contacting, in a reactor system, a hydrocarbon-containing feedstock with the heterogeneous catalyst composition of claim 1 to generate olefinic compounds.

9. The process of claim 8, which is an exothermic process.

10. The process of claim 8, wherein the contacting in the reactor system is conducted at a temperature of about 750° C. or less.

11. The process of claim 8, wherein the process is carried out at a pressure of about 20 atm or less.

12. The process of claim 8, wherein the olefinic compounds comprise light olefins, α-olefins, and terminal dienes, selected from the group consisting of ethene, propene, 1-butene, 2-methyl-but-1-ene, 1-n-pentene, 1-n-hexene, 2-methyl-pent-1-ene, 3-methyl-pent-1-ene, 1,3-butadiene, 1,3-pentadiene, 1,4-pentadiene, 1,3-hexadiene, 1,4-hexadiene, and 1,5-hexadiene.

13. The process of claim 8, wherein the hydrocarbon-containing feedstock comprises at least one light alkane.

14. The process of claim 8, wherein the contacting is in the presence of:
an oxygen source, wherein the oxygen source comprises a purified O₂ stream, an air stream, or a mixture thereof; and
optionally, a diluent selected from the group consisting of nitrogen, argon, or helium.

15. The process claim 8, wherein the reactor system comprises a single reactor or at least a first reactor and a second reactor connected in a continuous loop for catalyst circulation.

16. The process of claim 14, wherein the reactor system comprises a single reactor, and the heterogeneous catalyst composition is contacted sequentially, first with the hydrocarbon-containing feedstock, then with the oxygen source.

17. A process for preparing a heterogeneous catalyst composition, the process comprising:
combining a porous mixed metal oxide support with at least one metal catalyst precursor to form a catalyst precursor mixture,
wherein the at least one metal catalyst precursor comprises at least one metal compound selected from the group consisting of transition metal compounds, rare-earth metal compounds, or a mixture thereof; and
heating the catalyst precursor mixture to a temperature of about 390° C. to about 750° C. to form the heterogeneous catalyst composition of claim 1.

18. The process of claim 17, further comprising:
preparing the heterogeneous catalyst composition outside of a reactor system; and
loading the heterogeneous catalyst composition into the reactor system.

19. The process of claim 18, wherein the preparing comprises:
combining a porous mixed metal oxide with a metal catalyst precursor to form a catalyst precursor mixture,
wherein the catalyst precursor mixture comprises at least one metal compound selected from the group consisting of transition metal compounds, rare-earth metal compounds, or a mixture thereof; and
heating the catalyst precursor mixture to a temperature of about 390° C. to about 750° C. to form the heterogeneous catalyst composition.

20. The process of claim 17, further comprising:
preparing the heterogeneous catalyst composition inside a reactor system;
loading the catalyst precursor mixture into the reactor system;
wherein the catalyst precursor mixture comprises:
the porous mixed metal oxide support; and
the at least one metal catalyst precursor, comprising the at least one metal compound selected from transition metal compounds, rare-earth metal compounds, or a mixture thereof; and
heating at a temperature of about 390° C. to about 750° C.
